(19) United States
(12) Patent Application Publication
(10) Pub. No.: US 2022/0175446 A1
(43) Pub. Date: Jun. 9, 2022

(54) DEVICES, SYSTEMS, AND METHODS FOR TARGETED ABLATION

(71) Applicant: Covidien LP, Mansfield, MA (US)

(72) Inventors: Lisa M. Quealy, Dracut, MA (US); Nikolai D. Begg, Wellesley, MA (US)

(21) Appl. No.: 17/534,130

(22) Filed: Nov. 23, 2021

Related U.S. Application Data
(60) Provisional application No. 63/123,332, filed on Dec. 9, 2020.

(51) Int. Cl. A61B 18/14; A61B 18/12

(52) U.S. Cl. CPC A61B 18/1492; A61B 2018/00577; A61B 18/1206

(57) ABSTRACT

A method of surgery includes determining a target zone of tissue, adjusting settings of a treatment device based on the target zone of tissue, positioning the treatment device adjacent the target zone of tissue, deploying the treatment device such that the treatment device defines a deployed configuration in accordance with the settings, and treating the target zone of tissue using the treatment device. An ablation system includes a housing, an elongated body extending distally from the housing, and a plurality of probes deployable from the elongated body. At least one control is configured to adjust settings associated with at least one probe such that, upon deployment of the plurality of probes, the plurality of probes define a deployed configuration in accordance with the settings.

This application claims the benefit of, and priority to, U.S. Provisional Patent Application No. 63/123,332, filed on Dec. 9, 2020, the entire contents of which are hereby incorporated herein by reference.

### FIELD

The present disclosure relates to energy-based tissue treatment and, more particularly, to devices, systems, and methods facilitating targeted ablation of tissue, e.g., within the uterus.

### BACKGROUND

Disease conditions affecting the uterus include fibroids, polyps, endometriosis, adenomyosis, endometrial hyperplasia, and cancer. Fibroids are benign tumors of the uterus and are among the most common disease conditions affecting the uterus. In fact, fibroid affect up to 30% of women of childbearing age and can cause significant symptoms such as pain, discomfort, mennorhagia, pressure, anemia, compression, infertility, and miscarriage. Fibroids may be located, for example, in the myometrium, adjacent to the endometrium, or in the outer layer of the uterus. Disease conditions such as fibroids are treated in numerous ways, including via ablating diseased tissue.

### SUMMARY

As used herein, the term “distal” refers to the portion that is being described which is farther from an operator (whether a human surgeon or robotic device), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is a method of surgery including determining a target zone of tissue, adjusting settings of a treatment device based on the target zone of tissue, positioning the treatment device adjacent the target zone of tissue, deploying the treatment device such that the treatment device defines a deployed configuration in accordance with the settings, and treating the target zone of tissue using the treatment device.

In an aspect of the present disclosure, deploying the treatment device includes deploying a plurality of probes from the treatment device to the target zone of tissue. In such aspects, treating the target zone of tissue may include energizing at least one probe of the plurality of probes to supply energy to tissue, e.g., to ablate tissue.

In another aspect of the present disclosure, the settings influence at least one of a deployed extent, a deployed orientation, or a deployed position of at least one probe of the plurality of probes.

In still another aspect of the present disclosure, the target zone of tissue is determined at least partially from an image or model of tissue. The target zone of tissue may be determined, using the image or model, by a software application or may be user-selected.

In yet another aspect of the present disclosure, the settings are automatically adjusted based on the target zone of tissue.

An ablation system provided in accordance with aspects of the present disclosure includes a housing and an elongated body extending distally from the housing. The elongated body includes an outer tube having an internal passageway and at least one opening defined through the outer tube. A plurality of probes is disposed within the internal passageway of the outer tube. The plurality of probes is deployable from a retracted position, wherein the plurality of probes is substantially disposed within the outer tube, towards a deployed position, wherein the plurality of probes extends through the at least one opening and from the outer tube. At least one control is configured to adjust settings associated with at least one probe of the plurality of probes such that, upon deployment of the plurality of probes from the retracted position towards the deployed position, the plurality of probes define a deployed configuration in accordance with the settings.

In an aspect of the present disclosure, the system further includes at least one actuator disposed on the housing and configured to deploy the plurality of probes from the retracted position towards the deployed position.

In another aspect of the present disclosure, the system further includes at least one driver operably coupled between the at least one actuator and the plurality of probes. The at least one driver is configured to deploy the plurality of probes from the retracted position towards the deployed position in response to actuation of the at least one actuator.

In still another aspect of the present disclosure, actuation of the at least one actuator translates the at least one driver to deploy the plurality of probes to the deployed position, e.g., to deploy the probes distally from the outer tube.

In yet another aspect of the present disclosure, actuation of the at least one actuator rotates the at least one driver to deploy the plurality of probes to the deployed position, e.g., to deploy the probes radially outwardly from the outer tube.

In still yet another aspect of the present disclosure, the elongated body further includes a plurality of inner sleeves disposed within the outer tube. Each probe of the plurality of probes is received within one of the inner sleeves of the plurality of inner sleeves. In such aspects, in the retracted position, each probe of the plurality of probes may be substantially disposed within and constrained by a corresponding inner sleeve of the plurality of inner sleeves. In the deployed position, each probe of the plurality of probes may extend from the corresponding inner sleeve and return towards an unconstrained position.

In another aspect of the present disclosure, the at least one control is configured to adjust the settings associated with the at least one probe by adjusting at least one of: a position of the at least one probe, an orientation of the at least one probe, or an arrangement between the at least one probe and at least one other probe.

In another aspect of the present disclosure, the at least one control is disposed on the housing. Alternatively, the at least one control may be remote.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1A is a side view of an ablation device in accordance with the present disclosure, disposed in a retracted position with a distal portion enlarged;

FIG. 1B is a side view of the ablation device of FIG. 1A, disposed in a deployed position with the distal portion enlarged;

FIG. 2 is a transverse, cross-sectional view taken across section line “2-2” of FIG. 1A;

FIG. 3A is a side view of a proximal portion of the ablation device of FIG. 1A including a plurality of mechanical setting controls;

FIG. 3B is a side view of a proximal portion of the ablation device of FIG. 1A including an electrical setting control system;

FIG. 4 is an illustration of a surgical system in accordance with the disclosure and configured for use with the ablation device of FIG. 1A;

FIG. 5 is a block diagram illustrating a portion of the surgical system of FIG. 4;

FIG. 6 illustrates the ablation device of FIG. 1A disposed in the deployed position and positioned within a uterus for treating a target tissue;

FIG. 7 illustrates the ablation device of FIG. 1A disposed in the deployed position and positioned within a uterus for treating another target tissue;

FIG. 8A is a side view of another ablation device in accordance with the present disclosure, disposed in a retracted position with a distal portion enlarged;

FIG. 8B is a side view of the ablation device of FIG. 8A, disposed in a deployed position with the distal portion enlarged;

FIG. 9 illustrates the ablation device of FIG. 8A disposed in the deployed position and positioned within a uterus for treating a target tissue; and

FIG. 10 is a schematic illustration of a robotic surgical system configured for use in accordance with the present disclosure.

### DETAILED DESCRIPTION

The present disclosure is directed towards energy-based tissue treatment and, more particularly, to devices, systems, and methods facilitating targeted ablation of tissue, e.g., within the uterus. Although detailed herein with respect to intrauterine ablation, the aspects and features of the present disclosure are equally applicable for targeted ablation of other organs and/or tissues.

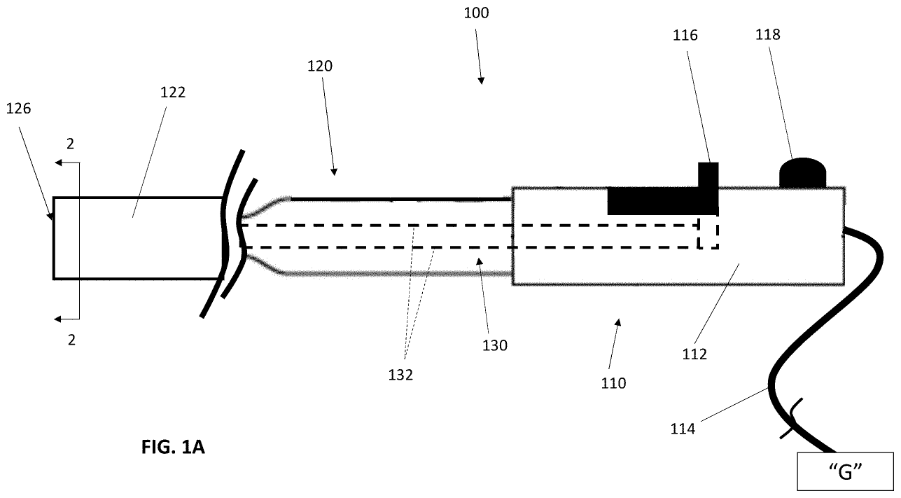

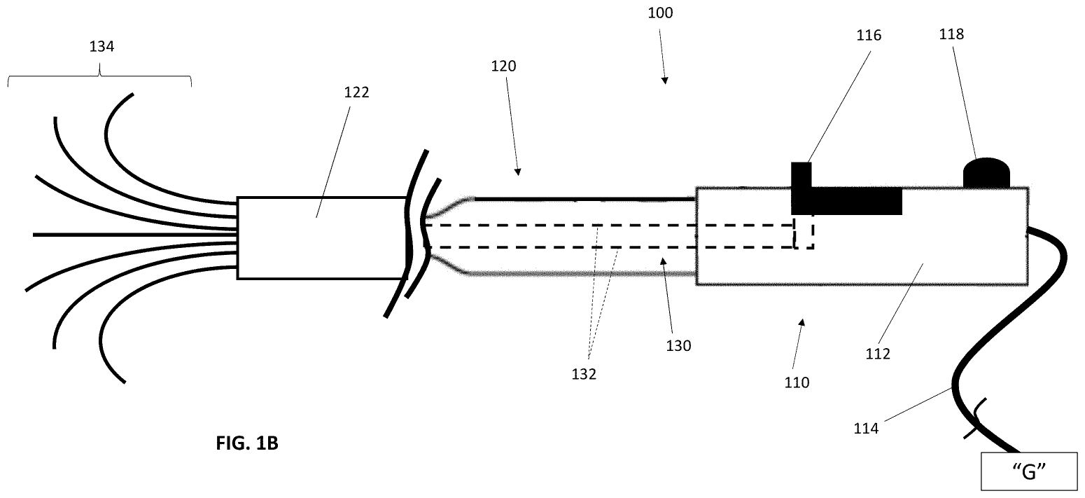

Turning to FIGS. 1A and 1B, an ablation device 100 provided in accordance with the present disclosure generally includes a handle assembly 110, an elongated body 120 extending distally from handle assembly 110, and a deployable assembly 130. Handle assembly 110 includes a housing 112 configured to be grasped and manipulated by a user, a cable 114 extending proximally from housing 112 to connect ablation device 100 to a generator “G, one or more actuators 116 configured to enable selective transitioning of deployable assembly 130 between a retracted position (FIG. 1A) and a deployed position (FIG. 1B), and an activation button 118. Although actuator 116 is illustrated as a slide knob for manual deployment of deployable assembly 130, any other suitable actuator for manual deployment of deployable assembly 130, e.g., a pivoting trigger, rotation wheel, plunger, etc., may be provided. As an alternative to manual deployment, actuator 116 may be any suitable electronic actuator, e.g., e.g., a switch, push-button, Graphical User Interface (GUI), etc., electrically coupled to a motor (not shown) disposed within housing 112 for selective powered deployment of deployable assembly 130. In either manual or powered configurations, plural actuators 116 may be provided to enable selective deployment of deployable assembly 130 to achieve a desired configuration, as detailed below. Activation button 118 is configured to enable the selectively activation of ablation device 100, e.g., to supply energy from generator “G” to deployable assembly 130 in the deployed position thereof (FIG. 1B) to treat tissue, as also detailed below.

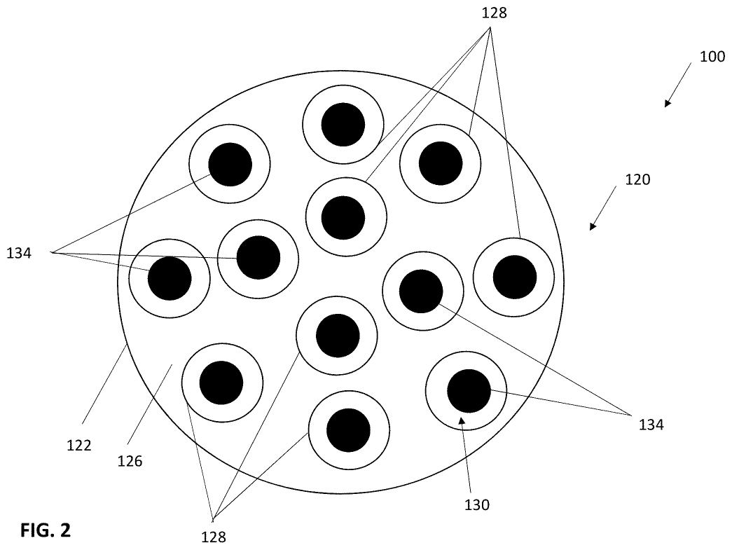

Elongated body 120, as noted above, extends distally from handle assembly 110. Elongated body 120 includes an outer tube 122 that may define a substantially constant outer diameter along its length, a continuously tapered diameter along at least a portion of its length, a step-wise tapered diameter along at least a portion of its length, or any other suitable configuration. Further, outer tube 122 of elongated body 120 may include one or more sections that are straight, pre-bent, rigid, flexible, malleable, and/or articulatable. Outer tube 122 is configured to extend through a natural orifice and/or surgically created opening into an internal surgical site. For example, outer tube 122 may be configured to extend transvaginally through the cervix and into the uterus, although other configurations are also contemplated. Outer tube 122 defines an interior longitudinal passageway 124 (FIG. 2) and one or more distal openings 126 in communication with the interior longitudinal passageway 124 (FIG. 2). The one or more distal openings 126 may be defined at the distal end face of outer tube 122 (as shown), and/or may be defined transversely through a side wall of outer tube 122 at a distal end portion thereof.

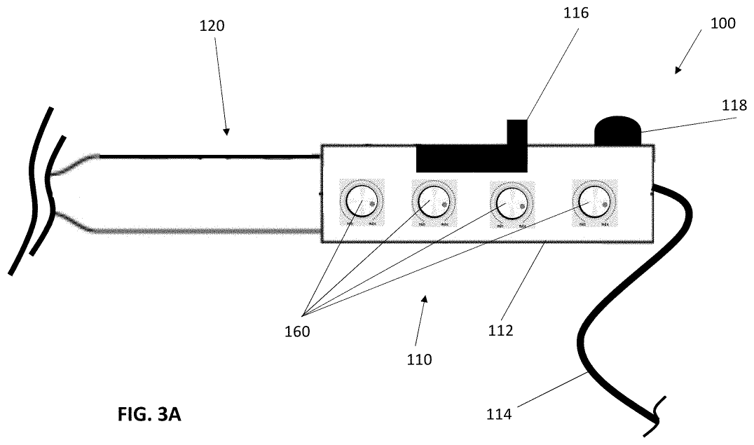

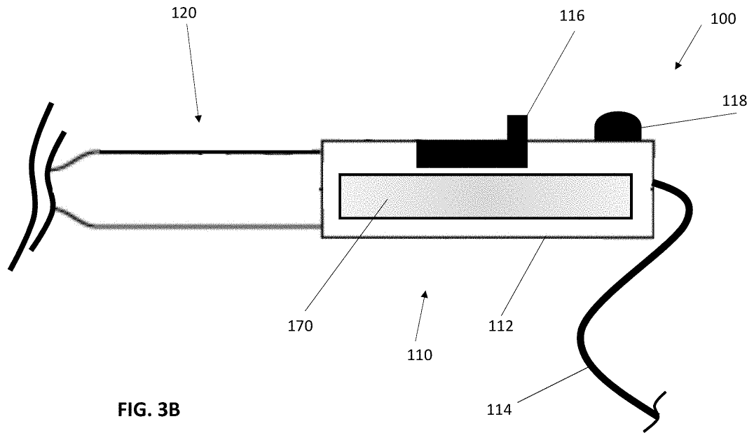

With additional reference to FIG. 2, elongated body 120 further includes a plurality of inner sleeves 128 disposed within longitudinal passageway 124 of outer tube 122 and extending at least partially therethrough. Inner sleeves 128 may be fixed relative to outer tube 122. Alternatively, inner sleeves 128 may be rotatable within and relative to relative to outer tube 122 individually, collectively, or in groups, and/or longitudinally slidable within and relative to outer tube 122 individually, collectively, or in groups. In configurations where inner sleeves 128 are rotatable and/or slidable relative to outer tube 122, such movement may be achieved manually, e.g., via one or more mechanical controls 160 (FIG. 3A) coupled to handle assembly 110, or may be powered, e.g., via one or more electrical controls 170 (FIG. 3B) disposed on handle assembly 110 associated with one or more motors (not shown) disposed within handle assembly 110. Movement of some or all of inner sleeves 128 may be accomplished independently of the deployment of deployable assembly 130 such that, as detailed below, inner sleeves 128 may be moved into a desired configuration to facilitate deployable assembly 130 achieving a desired deployed configuration upon deployment thereof In some configurations, inner sleeves 128 are substantially rigid and extend in substantially parallel orientation relative to one another, although other configurations are also contemplated. Inner sleeves 128 may be electrically insulative, coated with an electrically insulative materially (internally and/or externally), or otherwise configured. Likewise, outer tube 122 may be electrically insulative, coated with an electrically insulative materially (internally and/or externally), or otherwise configured.

Deployable assembly 130, as noted above, is selectively transitionable between a retracted position (FIG. 1A) and a deployed position (FIG. 1B). Deployable assembly 130 is operably coupled to the one or more actuators 116 within handle assembly 110 and extends distally therefrom through elongated body 120. Deployable assembly 130 includes one or more proximal drivers 132 and a plurality of distal probes 134. More specifically, each proximal driver 132 supports one or more distal probes 134. Each proximal driver 132 is operably coupled to one of the actuators 116 such that actuation of an actuator 116, e.g., sliding of the slide knob between a more-proximal position (FIG. 1A) and a more-distal position (FIG. 1B), translates the corresponding proximal driver(s) 132 between a more-proximal position and a more-distal position to thereby move the corresponding distal probe(s) 134 between a retracted position, corresponding to the retracted position of deployable assembly 130 (FIG. 1A), and a deployed position, corresponding to the deployed position of deployable assembly 130 (FIG. 1B).

In aspects wherein one actuator 116 is operably coupled to multiple proximal drivers 132 and/or wherein one proximal driver 132 supports multiple distal probes 134, selective adjustment between the multiple proximal drivers 132 of each corresponding actuator 116 and/or between the multiple distal probes 134 of each corresponding proximal driver 132 may be achieved, e.g., manually via one or more mechanical controls 160 (FIG. 3A) coupled to handle assembly 110 or powered via one or more electrical controls 170 (FIG. 3B) disposed on handle assembly 110 associated with one or more motors (not shown) disposed within handle assembly 110. In this manner, variation in the deployment of distal probes 134 may be achieved even where some of the distal probes 134 share a common proximal driver 132 and/or actuator 116.

Continuing with reference to FIGS. 1A-2, each distal probe 134 extends through one of the inner sleeves 128 of elongated body 120 and is selectively deployable therefrom. Distal probes 134 may be rotationally fixed relative to the corresponding inner sleeves 128 or may be rotatable relative thereto. In either configuration, in the retracted position of deployable assembly 130 (FIG. 1A), distal probes 134 are substantially disposed within the corresponding inner sleeves 128 and constrained thereby such that distal probes 134 substantially assume the configurations of the corresponding inner sleeves 128. In some aspects, inner sleeves 128 are substantially linear and, thus, distal probes 134 define linear configurations in the retracted position (FIG. 1A). Distal probes 134 are formed from resilient, shape memory, or other suitable material(s) such that, as distal probes 134 are deployed from (and, thus, no longer constrained by) inner sleeves 128, distal probes 134 return towards a pre-bent “home” configuration. In aspects, each distal probe 134 is configured to assume a radiused curvature upon deployment from the corresponding inner sleeve 128. The distal probes 134 may be configured to collectively define a plurality of radiused curves extending radially outwardly from a longitudinal axis of elongated body 120 in at least one configuration of the deployed position of deployable assembly 130 (see FIG. 1B). Other suitable “home” configurations of the distal probes 134, similar or different from one another, are also contemplated.

The length of deployment of each distal probe 134 in the deployed position depends on the extent to which each distal probe 134 is deployed from the corresponding inner sleeves 128 (and, in aspects, the position of the inner sleeves 128 relative to outer tube 122). That is, the more a distal probe 134 is advanced from its inner sleeve 128, larger portion thereof that is able to return towards the “home” configuration and, thus, the larger the arc of curvature (or other length of deployment) that is achieved for that distal probe 134. The deployment of the distal probes 134 may be varied individually, collectively, or in groups to achieve a desired amount of deployment for each of the distal probes 134. This variation in this relative deployment between the distal probes 134 may be achieved by: deploying the actuators 116 different amounts (in configurations where multiple actuators 116 are provided); advancing/retracting some or all of the inner sleeves 128 relative to the corresponding distal probes 134, e.g., prior to deployment; adjusting the operable coupling between the proximal drivers 132 and corresponding actuator 116 (where one actuator 116 is operably coupled to multiple proximal drivers 132); adjusting the operably coupling between the distal probes 134 and corresponding proximal driver 132 (where one proximal driver 132 supports multiple distal probes 134); combinations thereof; or in any other suitable manner.

The direction of curvature (or other deployment) of each distal probe 134 upon deployment depends on the orientations of the distal probes 134 relative to the corresponding inner sleeves 128 (e.g., where distal probes 134 are rotatable within inner sleeves 128) and/or the orientations of the inner sleeves 128 relative to outer tube 122 (e.g., where distal probes 134 are rotatably fixed relative to inner sleeves 128). These orientations may be changed by rotating the distal probes 134 and/or inner sleeves 128, individually, collectively, or in groups.

The relative spacing between each distal probe 134 and the arrangement of some or all of the distal probes 134 upon deployment depends on the positioning of the corresponding inner sleeves 128 within outer tube 122. Thus, by moving inner sleeves 128 relative to one another and/or outer tube 122, a desired spacing and/or arrangement can be achieved.

Referring also to FIGS. 3A and 3B, as noted above, one or more mechanical controls 160, e.g., dials, sliders, triggers, etc., disposed on handle assembly 110 may be provided to enable selective adjustment of some or all inner sleeves 128, selective adjustment of some or all proximal drivers 132, and/or selective adjustment of some or all distal probes 134. Additionally or alternatively, one or more electrical controls 170 (FIG. 3B), e.g., a touch-screen GUI, disposed on handle assembly 110 and associated with one or more motors (not shown) disposed within handle assembly 110 may be provided to enable selective adjustment of some or all inner sleeves 128, selective adjustment of some or all proximal drivers 132, and/or selective adjustment of some or all distal probes 134. It is contemplated that the above adjustments be performed prior to deployment of deployable assembly 130 such that, upon deployment to the deployed position, a desired configuration of the plurality of distal probes 134 is achieved. However, additional or alternative adjustment may be made after deployment, e.g., to reposition some or all of the distal probes 134 in the deployed position of deployable assembly 130. Further, the distal probes 134 may be deployed individually or in groups with adjustment before or after one or more deployments, to achieve a desired configuration of the plurality of distal probes 134 once all are deployed.

Achieving the desired configuration of distal probes 134 in the deployed position of deployable assembly 130 may be facilitated by selectively actuating the one or more actuators 116. Further, rather than making adjustments at ablation device 100 itself, adjustment may be made at a fixture device (not shown) configured to adjust ablation device 100 according to inputs provided thereto, adjustment may be made at generator “G” to signal ablation device 100 to make the appropriate adjustments based on the inputs provided thereto, adjustment may be made at a remote device operably connected to ablation device 100 and/or generator “G” to signal ablation device 100 to make the appropriate adjustments based on the inputs provided thereto, or adjustment may be made in any other suitable manner. Regardless of the particular manner of adjustment, the above-detailed configuration enables customization of the deployment depth, orientation, spacing, and arrangement of distal probes 134 of deployable assembly 130 to achieve a desired configuration, thus facilitating targeted ablation of any shape ablation zone of tissue.

Referring back to FIGS. 1-2B, distal probes 134 may define sharpened tips to facilitate penetration into tissue, may define blunt tips configured to maintain contact with a surface of tissue, or may define any other suitable configuration. Distal probes 134 may be configured as electrosurgical electrodes configured to deliver Radio-Frequency (RF) energy to tissue to treat, e.g., ablate, tissue. In such configurations, each distal probes 134 may define a bipolar configuration wherein each distal probe 134 includes one or more positive electrode portions and one or more negative electrode portions to enable the conduction of energy between the positive and negative electrode portions and through tissue to treat, e.g., ablate, tissue. Alternatively, distal probes 134 may collectively define a bipolar configuration wherein some distal probes 134 function as the positive electrodes while others function as the negative electrodes to enable the conduction of energy between the positive and negative electrodes and through tissue to treat, e.g., ablate, tissue. As an alternative to bipolar electrosurgical configurations, monopolar electrosurgical configurations are also contemplated, e.g., wherein distal probes 134 are configured as active electrodes for conducting energy to tissue that is returned via a remote return device (not shown), e.g., a return pad. Outer tube 122 and/or inner sleeves 128 may alternatively or additionally serve as the negative or return electrodes.

Rather than electrosurgical energy, distal probes 134 may be configured as microwave probes configured to delivery microwave energy, ultrasound probes configured to deliver ultrasound energy, thermal probes configured to deliver thermal energy, cryogenic probes configured to deliver cryogenic energy, or other suitable probes configured to deliver outer suitable forms of energy to tissue to treat, e.g., ablate, tissue. Cable 114 connects ablation device 100 to a suitable generator, e.g., generator “G,” to enable the supply of energy to distal probes 134 for treating tissue therewith. Activation button 118 enables the selective activation and/or deactivation of the supply of energy to distal probes 134. The ON/OFF, intensity, duration, etc. of energy supplied to distal probes 134 may be collectively controlled, individually controlled, or controlled in groups of distal probes 134. As such, in addition to achieving a desired mechanical arrangement of distal probes 134 in the deployed position of deployable assembly 130, as detailed above, a desired energy-applying arrangement can also be achieved, thus further facilitating targeted ablation of tissue. The selection of which distal probes 134 ere energized and the intensity, duration, etc. of such energization may be made at the generator, e.g., generator “G,” via one or more of the mechanical controls 160 (FIG. 3A), and/or via one or more of the electrical controls 170 (FIG. 3B).

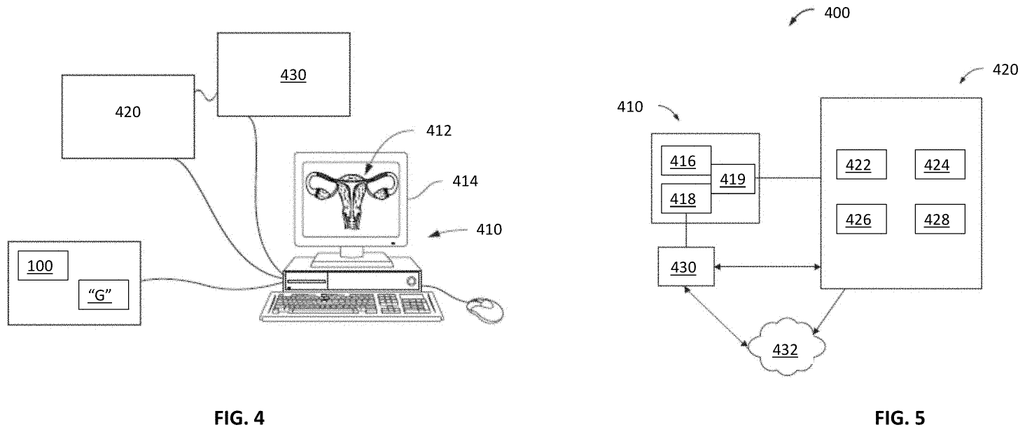

Turning to FIGS. 4 and 5, a system 400 provided in accordance with the present disclosure includes a computer 410 and a user interface 412 displayed on a suitable display 414 associated with computer 410 or any suitable monitoring equipment, e.g., an operating room monitor. Although illustrated as a desktop computer in FIG. 4, computer 410 may be any suitable computing device, such as a desktop computer, laptop computer, tablet, smartphone, server, etc. System 400 further includes a Hospital Information System (HIS) 420, a synthesizer 430, and an operative surgical system such as, for example, ablation device 100 and generator “G.” Computer 410 includes one or more processors 416 associated with one or more memories 418. Memory(s) 418 may include any non-transitory computer-readable storage media for storing one or more software applications that are executable by processor 416. A network module 419 of computer 410 enables communication between computer 410 and a network to which HIS 420 and synthesizer 430 are also connected.

HIS 420 may interface with a Picture Archiving and Communication System (PACS) 422, a Radiology Information System (RIS) 424, an Electronic Medical Records System (EMR) 426, and/or a Laboratory Information System (LIS) 428. PACS 422 stores and/or archives images of patients obtained from imaging systems such as, for example, X-ray CT, computerized axial tomography (CAT) scan, positron emission tomography (PET), single-photon emission CT (SPECT), Magnetic Resonant Imaging (MRI), Ultrasound (US), etc. RIS 424 complements HIS 420 and PACS 422 and serves as an electronic management system for an imaging department of a hospital, e.g., allowing a clinician to access digital images of a patient and to associate patient information from EMR 426 with the digital images stored in PACS 422. LIS 428 supports data exchange between a hospital laboratory and HIS 420 and, in particular, EMR 426.

Synthesizer 430 includes a software application stored in a memory, e.g., a memory 418, a memory of synthesizer 430, or another suitable memory, that is executable by a processor, e.g., processor 416, a processor of synthesizer 430, or another suitable processor. The software application of synthesizer 430 enables a clinician to access HIS 420 through network module 419 of computer 410 or via any other suitable computing device. More specifically, synthesizer 430 communicates with HIS 420 and provides a medium by which the clinician is able to gather data and utilize such data to, for example, pre-operatively determine the target location(s), tissue(s), etc. to be treated, e.g., ablated. Synthesizer 430 may interface with a synthesizer cloud 432, e.g., using a hardwired connection or wirelessly, such that the synthesizer 430 may access HIS 420 remotely, e.g., via a device not connected to the intranet, or may interface directly with HIS 420 to provide local access, e.g., within the intranet.

With continued reference to FIGS. 4 and 5, using information gathered from HIS 420 and/or other sources, synthesizer 430 provides an image and/or produces a model of the target patient anatomy, e.g., the uterus “U,” for display on a user interface, e.g., user interface 412, to enable the clinician to visualize uterine features and structures. More specifically, pre-operative image data gathered from HIS 420 is processed by the software application of synthesizer 430 to generate a three-dimensional (3D) image or model of the patient's uterus “U” that is displayed to the clinician, e.g., on user interface 412.

The software application of synthesizer 430 may automatically select, utilizing information gleaned from the three-dimensional (3D) image or model and/or from EMR 424 of HIS 420, and display, e.g., on user interface 412, a suggested target ablation zone(s) on the 3D image/model. The clinician may move or otherwise modify the suggested target ablation zone(s) or provide a different target ablation zone(s). The target ablation zone(s) may then be further modified and, finally, set. Alternatively, the clinician may select the target ablation zone(s) without input from the software application of synthesizer 430.

Referring also to FIGS. 1A-2, based on the final target ablation zone(s) determined, the software application of synthesizer 430, a processor associated with generator “G,” and/or a processor associated with ablation device 100 may determine the mechanical arrangement of distal probes 134 in the deployed position of deployable assembly 130 and the energy-applying arrangement of distal probes 134 that best achieves ablation of the final target ablation zone(s). These arrangement settings may be provided to ablation device 100 and/or generator “G” for automatically implementing the determined settings, and/or may be displayed or otherwise output to act as a guide to enable a user to manually implement the determined settings (pre-operatively or during use). Further, in addition to the above-detailed arrangement settings, an insertion depth, e.g., the extent to which outer tube 122 extend into the uterus “U,” and the orientation thereof, may be determined, e.g., by the application of synthesizer 430, a processor associated with generator “G,” and/or a processor associated with ablation device 100, as the position and orientation of outer tube 122 within the uterus “U” may affect the other arrangement settings.

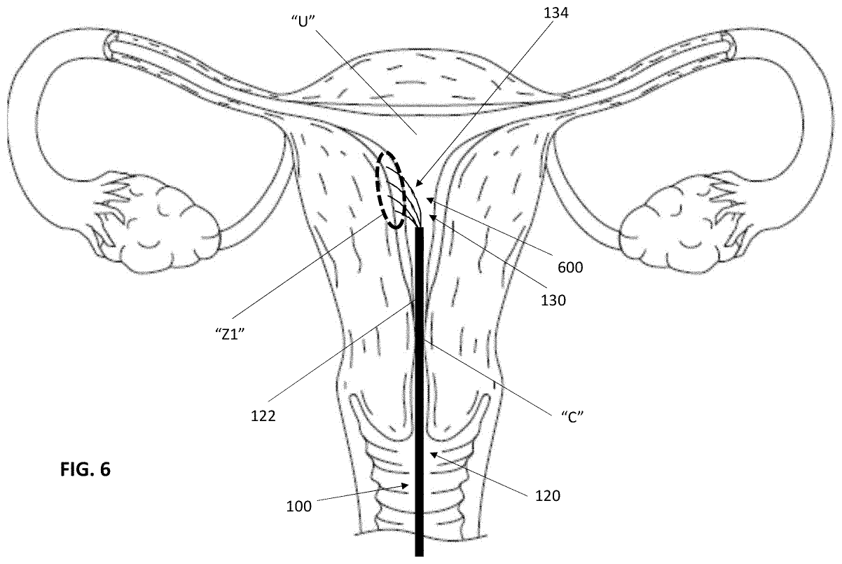

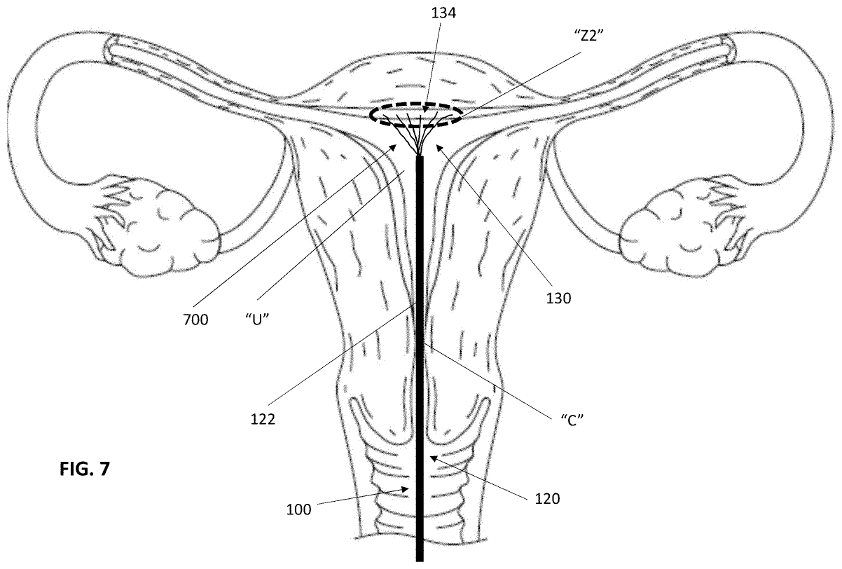

FIGS. 6 and 7 illustrate ablation device 100 extending transvaginally through the cervix “C” and into the uterus “U” with deployable assembly 130 disposed in the deployed position and distal probes 134 defining different configurations 600, 700, e.g., due to different arrangement settings implemented. Thus, ablation device 100 can be selectively deployed as desired to treat, e.g., ablate, tissue in a first ablation zone “Z1” using configuration 600 (FIG. 6) or to treat, e.g., ablate, tissue in a second ablation zone “Z2” using configuration 700 (FIG. 7). Although two different deployed configurations 600, 700 of the distal probes 134 are shown in FIGS. 6 and 7, respectively, it is understood that any suitable orientation, spacing/concentration, penetration depth, energy setting, etc. of distal probes 134 may be achieved to enable treatment of any ablation zone.

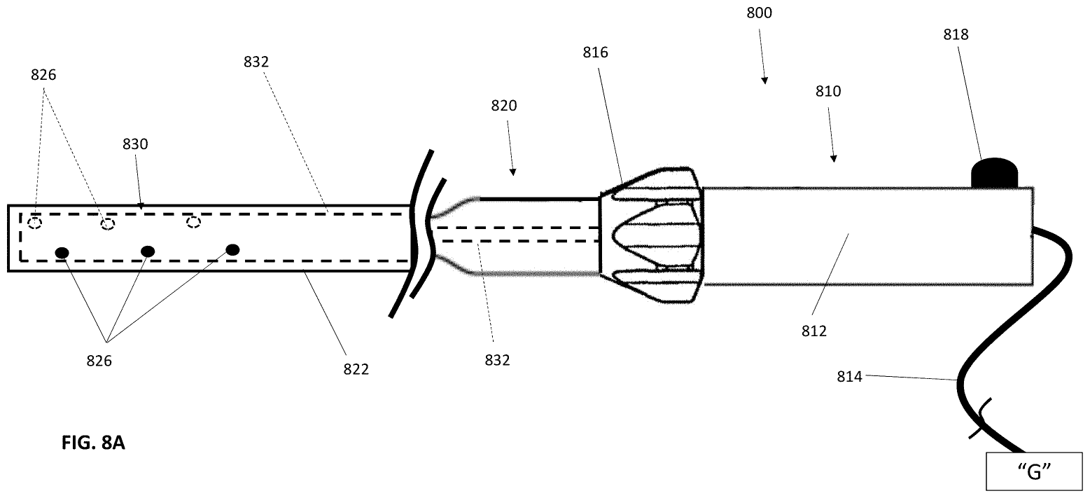

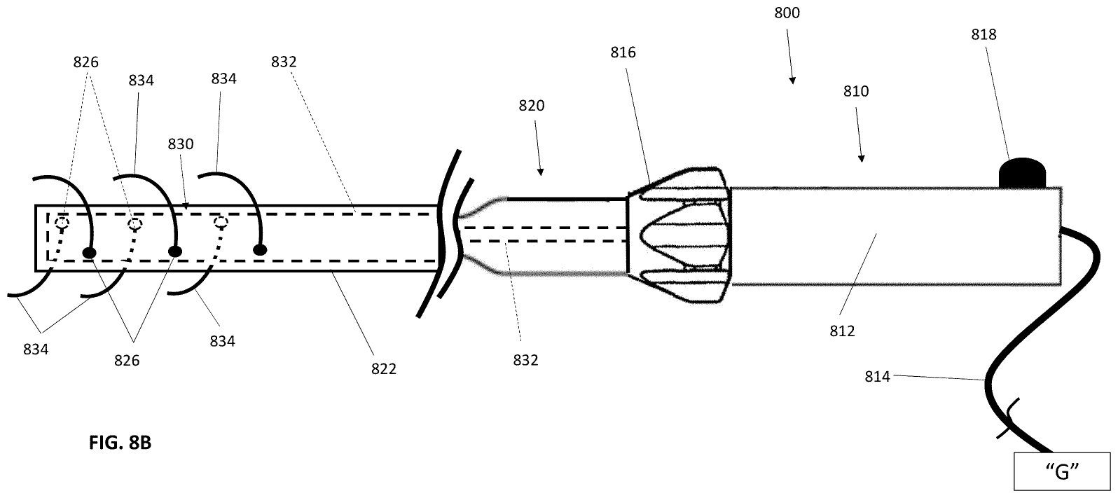

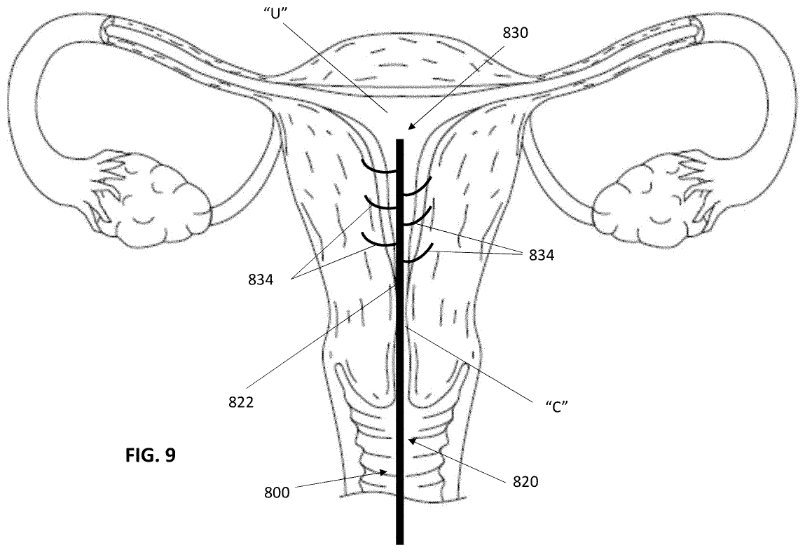

Referring to FIGS. 8A, 8B, and 9, another ablation device 800 is provided in accordance with the present disclosure. Ablation device 800 is similar to and may include any of the features of ablation device 100 (FIGS. 1A-2) detailed above and, thus, only differences therebetween are described in detail below while similarities are summarily described or omitted entirely. Ablation device 800 generally includes a handle assembly 810, an elongated body 820 extending distally from handle assembly 810, and a deployable assembly 830. Handle assembly 810 includes a housing 812 configured to be grasped and manipulated by a user, a cable 814 extending proximally from housing 812 to connect ablation device 800 to a generator “G,” one or more actuators 816 configured to enable selective transitioning of deployable assembly 830 between a retracted position (FIG. 8A) and a deployed position (FIG. 8B), and an activation button 818. Although the one or more actuators 816 are illustrated as a single rotation knob for manual deployment of deployable assembly 830, any other suitable actuator(s) for manual or powered deployment may be provided.

Elongated body 820 includes an outer tube 822 defining an interior longitudinal passageway (not shown) and a plurality of apertures 826 in communication with the interior longitudinal passageway. Apertures 826 extend through a side wall of outer tube 822 at a distal end portion thereof and may be arranged in a helical configuration about the outer periphery of the distal end portion of outer tube 822 or in any other suitable configuration or pattern. For example, apertures 826 may be arranged longitudinally in one or more lines, e.g., two diametrically opposed lines, more than two equally-spaced lines; arranged in a zig-zag or other suitable pattern; randomly arranged; etc.

Deployable assembly 830, as noted above, is selectively transitionable between a retracted position (FIG. 8A) and a deployed position (FIG. 8B). Deployable assembly 830 includes one or more rotational drivers 832 and a plurality of distal probes 834. The one or more rotational drivers 832 are operably coupled to the one or more actuators 816 and rotationally disposed within outer tube 822, e.g., such that rotation of actuator 816 rotates the rotational driver(s). In some configurations, the one or more rotational drivers 832 are rotationally and reciprocally disposed within outer tube 822, e.g., via a cam follower mechanism including a cam engaged within a helical groove. In aspects, one or more of the rotational drivers 832 is operably coupled to the one or more actuators 816 via suitable gearing such as, for example, a standard transmission, a Continuous Variable Transmission (CVT), etc., such that the rotation imparted from the one or more actuators 816 to the one or more rotational drivers 832 may be amplified and/or attenuated as desired. The rotation of the rotational drivers 832 may thus be adjusted collectively, individually, or in groups. One or more mechanical or electrical controls (not shown, see controls 160, 170 (FIGS. 3A and 3B)) may be provided for achieving a desired rotational output of the one or more rotational drivers 832 in response to an input to the one or more actuators 816. This adjustment enables customization of the deployment of distal probes 834 of deployable assembly 830 to achieve a desired configuration, thus facilitating targeted ablation of any suitable ablation zone of tissue.

Each distal probe 834 defines a fixed end and a free end. The fixed end of each distal probe 834 is secured to one of the rotational drivers 832. Distal probes 834 are wound, e.g., helically, radially, etc., about a corresponding one of the rotational drivers 832. Distal probes 834 may be wound in the same direction, although other configurations are also contemplated. The free end of each distal probe 834 is disposed in alignment (along the trajectory path of that distal probe 834) with a corresponding one of the apertures 826 defined through outer tube 822. Initially, the free ends of distal probes 834 are contained within, do not protrude from, or protrude minimally from outer tube 822. This position corresponds to the retracted position of deployable assembly 830 (FIG. 8A). Upon rotation of the one or more rotational drivers 832, the corresponding distal probes 834 are un-wound from about the rotational driver 832 thereof to extend through the apertures 826 of outer tube 822 to achieve the deployed position of deployable assembly 830 (FIG. 8B). In configurations where distal probes 834 are wound in the same direction, distal probes 834 define a similar trajectory upon exiting apertures 826 of outer tube 822, e.g., a helical configuration, although other configurations are also contemplated. As noted above, the amount of extension of individual, groups, or all distal probes 834 may be controlled via rotation of the one or more actuators 816 and/or based on adjustments to the gearing (or other suitable adjustment mechanism(s)).

With particular reference to FIG. 9, ablation device 800 is shown extending transvaginally through the cervix “C” and into the uterus “U” with deployable assembly 830 disposed in the deployed position and distal probes 834 extending radially outwardly from outer tube 822 to penetrate tissue. In this position, distal probes 834 may be energized to treat, e.g., ablate target tissue.

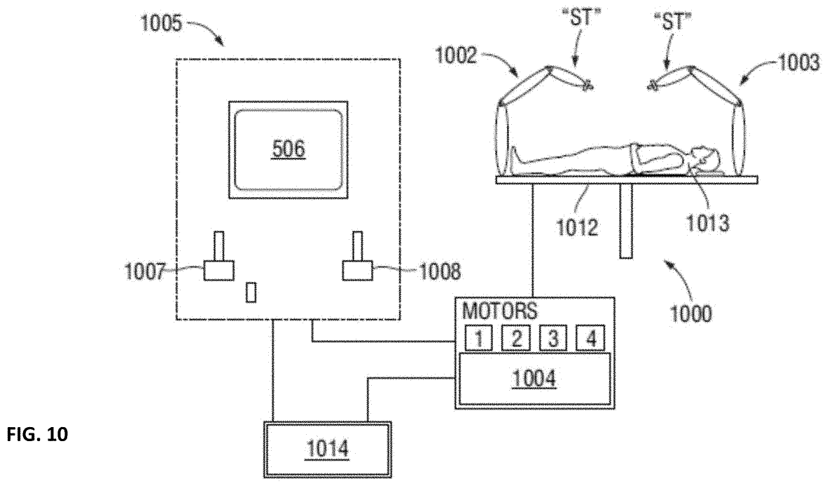

Turning to FIG. 10, a robotic surgical system 1000 configured for use in accordance with the present disclosure is shown. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person, e.g., a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and a mounted device which may be, for example, a surgical tool “ST.” The surgical tools “ST” may include, for example, any of the ablation devices of the present disclosure, a hysteroscope (or endoscope), an ultrasound probe. More specifically, with respect to the ablation devices detailed herein, the user-activation or actuation components are replaced with robotic inputs to enable a robot to provide the desired activation(s) and actuation(s) similarly as detailed above. That is, in robotic implementations, the ablation devices function similarly according to any of the aspects above except that the ablation devices are directly manipulated, activated, and/or actuated by a robot arm 1002, 1003 rather than a human surgeon.

Robot arms 1002, 1003 may be driven by electric drives, e.g., motors, connected to control device 1004. The motors, for example, may be rotational drive motors configured to provide rotational inputs to accomplish a desired task or tasks. Control device 1004, e.g., a computer, may be configured to activate the motors, in particular by means of a computer program, in such a way that robot arms 1002, 1003, and, thus, their mounted surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Control device 1004, more specifically, may control one or more of the motors based on rotation, e.g., controlling to rotational position using a rotational position encoder (or Hall effect sensors or other suitable rotational position detectors) associated with the motor to determine a degree of rotation output from the motor and, thus, the degree of rotational input provided. Alternatively or additionally, control device 1004 may control one or more of the motors based on torque, current, or in any other suitable manner.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. A method of surgery, comprising:
determining a target zone of tissue;
adjusting settings of a treatment device based on the target zone of tissue;
positioning the treatment device adjacent the target zone of tissue;
deploying the treatment device such that the treatment device defines a deployed configuration in accordance with the settings; and
treating the target zone of tissue using the treatment device.

2. The method according to claim 1, wherein deploying the treatment device includes deploying a plurality of probes from the treatment device to the target zone of tissue.

3. The method according to claim 2, wherein treating the target zone of tissue includes energizing at least one probe of the plurality of probes to supply energy to tissue.

4. The method according to claim 3, wherein the treatment device is an ablation device, and wherein treating the target zone of tissue includes ablating tissue.

5. The method according to claim 2, wherein the settings influence at least one of a deployed extent, a deployed orientation, or a deployed position of at least one probe of the plurality of probes.

6. The method according to claim 1, wherein the target zone of tissue is determined at least partially from an image or model of tissue.

7. The method according to claim 6, wherein the target zone of tissue is determined by a software application.

8. The method according to claim 6, wherein the target zone of tissue is user-selected.

9. The method according to claim 1, wherein the settings are automatically adjusted based on the target zone of tissue.

10. An ablation system, comprising:
a housing;
an elongated body extending distally from the housing, the elongated body including an outer tube having an internal passageway and at least one opening defined through the outer tube;
a plurality of probes disposed within the outer tube, the plurality of probes deployable from a retracted position, wherein the plurality of probes is substantially disposed within the outer tube, towards a deployed position, wherein the plurality of probes extends through the at least one opening and from the outer tube; and
at least one control configured to adjust settings associated with at least one probe of the plurality of probes such that, upon deployment of the plurality of probes from the retracted position towards the deployed position, the plurality of probes define a deployed configuration in accordance with the settings.

11. The ablation system according to claim 10, further comprising at least one actuator disposed on the housing and configured to deploy the plurality of probes from the retracted position towards the deployed position.

12. The ablation system according to claim 11, further comprising at least one driver operably coupled between the at least one actuator and the plurality of probes, wherein the at least one driver is configured to deploy the plurality of probes from the retracted position towards the deployed position in response to actuation of the at least one actuator.

13. The ablation system according to claim 12, wherein actuation of the at least one actuator translates the at least one driver to deploy the plurality of probes to the deployed position.

14. The ablation system according to claim 13, wherein the plurality of probes is deployed distally from the outer tube.

15. The ablation system according to claim 12, wherein actuation of the at least one actuator rotates the at least one driver to deploy the plurality of probes to the deployed position.

16. The ablation system according to claim 15, wherein the plurality of probes is deployed radially outwardly from the outer tube.

17. The ablation system according to claim 10, wherein the elongated body further includes a plurality of inner sleeves disposed within the outer tube, each probe of the plurality of probes received within one of the inner sleeves of the plurality of inner sleeves.

18. The ablation system according to claim 17, wherein, in the retracted position, each probe of the plurality of probes is substantially disposed within and constrained by a corresponding inner sleeve of the plurality of inner sleeves, and wherein, in the deployed position, each probe of the plurality of probes extends from the corresponding inner sleeve and returns towards an unconstrained position.

19. The ablation system according to claim 10, wherein the at least one control is configured to adjust the settings associated with the at least one probe by adjusting at least one of: a position of the at least one probe, an orientation of the at least one probe, or an arrangement between the at least one probe and at least one other probe.

20. The ablation system according to claim 1, wherein the at least one control is disposed on the housing.
